(19) United States
(12) Patent Application Publication
(10) Pub. No.: US 2003/0104070 A1
(43) Pub. Date: Jun. 5, 2003

(54) Coated potassium chloride granules and tablets

(76) Inventors: Bradley L. Christenson, Blaine, MN (US); Mark J. Pieloch, Lincoln, NE (US)

Correspondence Address: Paul W. Busse, FAEGRE & BENSON LLP, 2200 Wells Fargo Center, 90 South Seventh Street, Minneapolis, MN, 55402-3901, US

(21) Appl. No.: 10/040,070

(22) Filed: Oct. 23, 2001

(51) Int. Cl. A61K 33/14; A61K 9/16; A61K 9/50

(52) U.S. Cl. 424/495; 424/679

(57) ABSTRACT

This invention provides extended release potassium chloride granules consisting essentially of potassium chloride crystals having a mesh size of about 20-60 mesh that are coated only with ethylcellulose. The granules may be compressed into tablets that disintegrate rapidly in an aqueous environment to provide uniform dissolution of the potassium chloride. Processes to produce extended release granules without using surfactants, processing aids or other coating aids are also provided by this invention.

### BACKGROUND

This invention generally provides coated potassium chloride granules that may be used to make extended release potassium chloride tablets. Specifically, the present invention provides ethylcellulose-coated crystals of potassium chloride that may be orally administered to a patient requiring potassium supplementation. The coated potassium chloride granules provide extended release of the potassium chloride in the gastrointestinal tract that results in substantially less irritation to the gastric mucosa.

The administration of many diuretics, commonly used to treat patients having hypertension, increases the excretion of both sodium and potassium. The acute administration of such diuretics to a patient normally causes no problems. However, chronic administration of diuretics to some patients can result in the depletion of potassium from the patient, a condition known as hypokalemia. Potassium depletion may be accompanied by a reduced tolerance to carbohydrates and a deficiency in glycogen deposition. Further, vasopressin-resistant polyuria is another complication. A deficit of potassium also appears to increase the renal synthesis of prostaglandins, which in turn can decrease the permeability to water of the distal nephron and produce a diabetes insipidus-like syndrome.

In order to avoid these complications, supplemental potassium administration is typically needed. When potassium is taken along with a normal diet it is slowly absorbed from the intestinal tract. Following potassium distribution and uptake by the cells, the kidneys excrete an appropriate amount to maintain a proper balance. As a consequence of a large volume of distribution and a rapid response of the kidney, the extracellular and intracellular concentrations of potassium are normally maintained within relatively narrow limits.

When potassium is administrated as a drug, the factors that govern the rate and extent of its distribution are of major importance. It is not possible to increase the total cellular content of potassium significantly above normal. However, it is very easy to raise the extracellular concentration excessively. It is the concentration of potassium in the extracellular fluid that determines life-threatening toxicity.

It is well known that large doses of potassium chloride taken orally can cause gastrointestinal irritation, purging, weakness and circulatory disturbances. Since potassium depletion can cause problems for the patient, a controlled or extended release formulation of potassium chloride that replenishes potassium in an acceptable manner without undesirable side effects is desired.

In an attempt to meet the need for suitable formulations that may be used as a potassium supplement, a number of different dosage formulations have been developed. U.S. Pat. No. 4,352,791 reports a composition of potassium and a therapeutically acceptable salicylate salt of salicylic acid. U.S. Pat. No. 4,340,582 reports an enteric coated tablet that may include potassium chloride. U.S. Pat. No. 4,259,323 reports a potassium chloride emulsion. U.S. Pat. No. 4,259,315 reports a controlled release potassium dosage from gelatin capsules that contain a mixture of ethylcellulose-encapsulated potassium chloride and a hydrophilic surfactant. Film-coated tablets containing potassium chloride in a wax matrix (non-enteric coated) are marketed as a slowly available potassium source. U.S. Pat. No. 4,235,870 reports a slow release pharmaceutical composition of a combination of higher aliphatic alcohols and hydrated hydroxyalkyl cellulose. U.S. Pat. No. 4,863,743 reports a controlled release potassium chloride tablet made of potassium chloride crystals coated with higher molecular weight (measured viscosity greater than 40 cP in toluene/ethanol) ethylcellulose and hydroxypropylcellulose. U.S. Pat. No. 5,397,574 reports controlled release potassium chloride micropellets coated with lower molecular weight (measured viscosity less than 10 cP in toluene/ethanol) ethylcellulose and a plasticizer.

### SUMMARY OF THE INVENTION

This invention provides a potassium chloride granule that contains both crystals of potassium chloride and a thermoplastic cellulose ether that forms a coating on the crystals. No other agents, additives, surfactants, or coating and processing aids are used or included in the granule. In one embodiment of the invention the potassium chloride crystals have a size of about 20-60 mesh. In another embodiment the thermoplastic cellulose ether is ethylcellulose having a measured viscosity of about 20 cP.

This invention also includes an extended release tablet made of a plurality of the ethylcellulose-coated potassium chloride granules. The potassium chloride granules that are in the tablet are essentially free of surfactants or other processing additives and agents. The term “essentially free” indicates the absence of surfactants, additives, agents or coating and processing aids during the processing of the granules.

Further, the invention includes a process to produce ethylcellulose-coated potassium chloride granules comprising the steps of i) forming a fluidized bed of potassium chloride crystals at a dew point of about 10-20° C., ii) spraying the fluidized crystals with a mixture of only ethylcellulose, alcohol and water sufficient to coat the crystals, and iii) drying the coated crystals to remove the alcohol and water to provide ethylcellulose-coated potassium chloride granules. The alcohol solvent can be any of the low-boiling C₁-C₄ alcohols, such as ethyl alcohol or isopropyl alcohol, and preferably methyl alcohol

The invention also provides a process for producing ethylcellulose-coated granules in which no additives are required during the spray-coating step for the control of static buildup in the fluid bed processor. The process comprises the steps of i) forming a fluidized bed of potassium chloride crystals, ii) spraying the fluidized crystals with a mixture consisting of ethylcellulose, alcohol, and sufficient water to control the buildup of static charge to enable substantially complete coating of the crystals, and iii) drying the coated crystals to remove the alcohol and water to provide ethylcellulose-coated potassium chloride granules. A water content of approximately 0.5 to 2% by weight has been found to be satisfactory for the control of static buildup in the fluid bed processor.

### DETAILED DESCRIPTION OF THE INVENTION

The present invention provides an extended release potassium chloride tablet that includes ethylcellulose-coated potassium chloride crystals. No other surfactants or processing additives and agents are used in, or are part of, the coating.

Static buildup is typically controlled in fluid-bed processing by means of a processing additive. One undesirable consequence of excessive static buildup is that particulate material may stick to the walls of the processing chamber, which can result in reduced yield or efficiency. The material sticking to the walls will be incompletely coated and may not be included with the final product, leading to decreased yield. Material clinging to the chamber walls also complicates cleanup between processing runs. An additional consequence of the presence of excessive static is an increased risk of explosion. An explosion can be triggered by static discharge in the fluid bed environment, due to the large amounts of fine dust present and, in some cases, the presence of organic solvent vapors.

Some common additives that are used to control static buildup include magnesium stearate, titanium dioxide, and talc as well as commercially available products sold under the trade names STAT-LES by Walter G. Legge Company, Inc., Peekskill, N.Y., and LAROSTAT by PPG/Mazer Chemicals, Gurnee, Ill. Nonionic surfactants have also been used as additives for static control. Suitable nonionic surfactants possess a hydrophilic-lipophilic balance (HLB) number in the range 4-15. Examples of suitable nonionic surfactants include sorbitan monooleate (SPAN 80, HLB 4.3), polysorbate 60 (TWEEN 60, HLB 14.9) and polysorbate 80 (TWEEN 80, HLB 15.0). The compositions and processes of the present invention do not require additives for static control.

Plasticizers are another class of processing additives that are commonly used in the manufacturing process. A plasticizer can be used to change the flexibility, tensile strength or adhesion properties of a polymeric film. When used in a pharmaceutical coating, a plasticizer can be used to improve or optimize the friability of a dosage unit or the dissolution profile of a delayed-release or extended-release dosage unit. Some common plasticizers that find use in pharmaceutical coating applications include dibutyl phthalate, diethyl phthalate, triethyl citrate, tributyl citrate, acetylated monoglyceride, acetyl tributyl citrate, dimethyl phthalate, benzyl benzoate, propylene glycol, butyl and glycol esters of fatty acids, low-molecular-weight polyethylene glycols, refined mineral oils, glycerin, oleic acid, organic acid esters, stearyl alcohol, castor oil, corn oil and camphor. The compositions and processes of the present invention do not utilize plasticizers as additives for the thermoplastic polymer coating.

The active ingredient of the tablet provided by the present invention is potassium chloride. Potassium chloride is administered to a patient in such a manner in order to prevent or relieve potassium depletion and to avoid side effects. The potassium chloride tablets of the present invention may be co-administered with a diuretic.

Potassium chloride occurs in nature as the mineral sylvine or sylvite. Various industrial preparations of potassium chloride also exist. Further, there are numerous pharmaceutical potassium chloride preparations. Potassium chloride is a white crystal or crystalline powder having the following physical description: d 1.98; mp 773° C.; 1 gram dissolves in 2.8 ml water; 1.8 ml boiling water; 14 ml glycerol; about 250 ml alcohol; and is insoluble in ether and acetone.

In the present invention, potassium chloride crystals having a particle size distribution ranging from about 20-60 mesh are subjected to coating or encapsulation in ethylcellulose and then are subsequently compressed into the tablet.

By providing a suitable coating weight of the ethylcellulose, a thermoplastic cellulose ether polymer film may be formed on the crystals that remains intact in the stomach (and afterwards) but which is permeable to gastric fluids. The gastric fluids dissolve and leach out the potassium chloride contained in the coated crystals.

The thermoplastic polymer coating of ethylcellulose on the crystals makes up about 17% of the total weight of granules. Lesser amounts of ethylcellulose in the coating may lead to or cause the formation of bare spots on the potassium chloride crystals during the compression step, leading to undesirably rapid release of the potassium chloride in the body after oral administration. Greater amounts of ethylcellulose, as the thermoplastic polymer coating, may cause the potassium chloride to be released too slowly to be completely absorbed by the patient. In one embodiment, potassium chloride is coated with ethylcellulose having a suitable molecular weight such as ethylcellulose designated as having a product viscosity of 20 and sold under the trademark ETHOCEL by Dow Chemical Company, Midland, Mich. The numerical designations for ethylcellulose generally correspond to the viscosity of the product, with a greater numerical designation indicating a greater measured viscosity and higher molecular weight. The 20 designation corresponds to a viscosity of about 18-22 cP as measured in a 5% by weight solution in an 80% toluene-20% ethanol solvent at 25° C. in an Ubbelohde viscometer. The ethoxyl content for ETHOCEL Standard 20 Premium cellulose ether is about 48-49.5%.

The individual crystals of potassium chloride are coated with the appropriate materials, mixed with acceptable compression aids and disintegrants and then compressed into tablets. The tablets are compressed in a manner so as to allow the tablets to disintegrate relatively quickly upon contact with an aqueous environment into the individual coated granules, i.e., disintegration takes place in a short period of time after oral administration.

The manufacturing process utilized in the present invention applies a controlled and uniform coating, permitting uniform dissolution. Accordingly, the rapid disintegration and controlled dissolution of the tablets into individual granules and the controlled dissolution of the granules permit the peristaltic motion of the gut to distribute the coated granules over a wide surface area. As a result, concentrated quantities of potassium chloride do not come in contact with the gastrointestinal mucosa, thus reducing the undesired side effect of gastric ulcers.

The importance of potassium supplement therapy has been well established. Physicians need products for the prevention of hypokalemia during chronic diuretic therapy. Compliance is essential for patients undergoing this type of therapy. Potassium chloride is normally provided in relatively large oral dosages in the range of 2 to 4 grams daily. Because of the large amount of the potassium chloride which is provided to the patient, gastrointestinal irritation is common. This irritation can range from a slight discomfort to gastric ulcers. By including potassium chloride crystals in the granules in the manner indicated above and then compressing them in a conventional manner into tablets, the gastrointestinal irritation is alleviated or eliminated.

The recommended dose in most patients is 40 mEq per day in divided doses. In accordance with currently approved labeling a single dose of 20 mEq (or 2 doses of 10 mEq) should be taken twice daily in order to obtain a daily dose of 40 mEq. With the formulation provided by the present invention, suitable tablets will include a dose of 20 mEq so that the recommended effective amount of potassium per single dose would not be altered. The daily dose is achieved with one tablet twice daily thus facilitating compliance due to less individual units per dose. Alternatively, other dosage units having 10 mEq or 15 mEq are included in the present invention and provide flexibility in establishing a regimen that meets a patient's needs.

In severe cases of hypokalemia, higher doses (60-80 mEq) of potassium may be required to reduce the loss of potassium during high dose diuretic therapy. In such cases, the physician would have available a safe higher-strength tablet where, in his judgment, he is treating a patient with a compliance problem.

The tablets produced by the present invention disintegrate into numerous sub-units when placed in water or placed on an aqueous food. After being disintegrated into the sub-units or micro-pellets the potassium chloride of the present invention can be more easily administered to children and geriatric patients who often have difficulty in swallowing large tablets. The tablets may include conventional compression aids such as microcrystalline cellulose and disintegrants such as croscarmellose sodium. In addition, other additives may be beneficial. For example, magnesium stearate or stearic acid could be added as lubricating agent, if necessary, to the compositions and methods of the present invention.

The present invention is further described in the following non-limiting example.

### Comparative Example

Dosage proportional 10 mEq and 20 mEq tablets were compressed from a blend of materials listed in Table 1 which includes ethylcellulose-coated potassium chloride granules. The Tablet A formula for the coated potassium chloride granules included sorbitan monooleate (SORBITAN) added to the coating solution. The SORBITAN was added with small amounts of purified water to the coating solution during the coating process, to reduce static buildup. The added purified water was removed during the coating process, but the nonvolatile SORBITAN was retained in the polymeric coating. The Tablet B formula for the coated potassium chloride granules did not include SORBITAN.

TABLE 1

| | | |
|---|---|---|
| | Tablet A | Tablet B |
| | 10 mEq (wt %) | 10 mEq (wt %) |
| Ingredient | 20 mEq (wt %) | 20 mEq (wt %) |

| | | | | |
|---|---|---|---|---|
| Sorbitan | 0.4 | 0.4 | 0 | 0 |
| Monooleate | | | | |
| Potassium Chloride | 75.0 | 75.0 | 75.3 | 75.3 |
| Ethylcellulose | 15.4 | 15.4 | 15.5 | 15.5 |
| Microcrystalline | 8.7 | 8.7 | 8.7 | 8.7 |
| Cellulose | | | | |
| Croscarmellose | 0.5 | 0.5 | 0.5 | 0.5 |
| Sodium | | | | |
| Total mg/tablet | 1,000 | 2,000 | 996 | 1,992 |

A blend of the listed materials used to form a 400 kg lot was compressed into both 10 mEq and 20 mEq tablets to provide Tablet A in two dosages. The dissolution profiles for the two tablets are listed in Table 2. The dissolution specification for the 1, 2, 6 and 12 hour dissolution test points are: for one hour not less than 10% not more than 30%; for two hours not less than 20% not more than 60%; for six hours not less than 50% not more than 90%; and for twelve hours not less than 85%.

TABLE 2

| Tablet A | 1 Hour | 2 Hour | 4 Hour | 6 Hour | 8 Hour | 12 Hour |
|---|---|---|---|---|---|---|
| 10 mEq with | 19% | 34% | 61% | 79% | 91% | 100% |
| SORBITAN | | | | | | |
| 20 mEq with | 18% | 31% | 56% | 76% | 91% | 100% |
| SORBITAN | | | | | | |

A blend of materials listed in Table 1 was used to form 400 kg lot was compressed into both 10 mEq and 20 mEq tablets to provide Tablet B in two dosages. By increasing the amount of purified water added to the coating solution by about 2% by weight, the SORBITAN was removed from the coating solution while still controlling the amount of static charge present in a Wurster Coater during the coating procedure. The dissolution profiles for the two tablets are listed in Table 3.

TABLE 3

| Tablet B | 1 Hour | 2 Hour | 4 Hour | 6 Hour | 8 Hour | 12 Hour |
|---|---|---|---|---|---|---|
| 10 mEq | 24% | 42% | 67% | 86% | 98% | 104% |
| 20 mEq | 24% | 39% | 64% | 87% | 97% | 104% |

The revised Tablet B 10 mEq and 20 mEq tablets meet the same specification used for the Tablet A 10 mEq and 20 mEq tablets. The data also indicates that the extended release properties of both sets of tablets are substantially the same, even though the Tablet B set does not include SORBITAN.

Both Tablet A dosages and Tablet B dosages were made using known processes. Briefly, a solution of ethylcellulose in methyl alcohol and water (with and without SORBITAN, respectively) was sprayed onto potassium chloride crystals in a 32-inch Wurster coater. The process parameters used for the coater are listed in Table 4.

TABLE 4

| | |
|---|---|
| Bottom Plate Configuration | G upbed plate, B downbed plate |
| Partition Height | 25 mm |
| Spray Nozzle Size | 2.2 mm |
| Spray Rate/Nozzle | 600-1000 g/min |
| Atomization Air Volume | 50-60 cfm |
| Product Temperature | 45-60° C. |
| Process Air Volume | 2700-3700 cfm |

After coating, the potassium chloride granules were then dried and blended with microcrystalline cellulose and croscarmellose sodium and compressed into tablets using a rotary tablet press.

What is claimed is:

1. A granule consisting of:
(a) crystals of potassium chloride; and
(b) a thermoplastic cellulose ether.

2. The granule of claim 1, wherein the potassium chloride crystals are between about 20-60 mesh.

3. The granule of claim 1, wherein the thermoplastic cellulose ether is ethylcellulose.

4. The granule of claim 3, wherein the ethylcellulose has a viscosity between approximately 10-30 cP.

5. An extended release tablet comprising a plurality of granules consisting of potassium chloride crystals and a thermoplastic cellulose ether.

6. The tablet of claim 5, wherein the granules are essentially free of surfactants or processing aids and agents.

7. The tablet of claim 5, wherein the potassium chloride crystals comprise approximately 75.3% by weight based on the total weight of the tablet.

8. The tablet of claim 5, wherein the thermoplastic cellulose ether is ethylcellulose.

9. The tablet of claim 8, wherein ethylcellulose comprises approximately 15.5% by weight based on the total weight of the tablet.

10. A pharmaceutical dosage unit in tablet form comprising a plurality of granules having an internal core of potassium chloride and an external coating of ethylcellulose, wherein the granules are essentially free of surfactants or processing aids and agents.

11. The tablet of claim 10, wherein the core of potassium chloride comprises approximately 75.3% by weight based on the total weight of said tablet.

12. The tablet of claim 10, wherein the ethylcellulose comprises approximately 15.5% by weight based on the total weight of said tablet.

13. A process to produce ethylcellulose-coated potassium chloride granules comprising the steps of:
i) forming a fluidized bed of potassium chloride crystals at a dew point of about 10-20° C.,
ii) spraying the fluidized crystals with a mixture consisting of ethylcellulose, alcohol and water sufficient to coat the crystals, and
iii) drying the coated crystals to remove the water and alcohol to provide coated potassium chloride granules.

14. The process according to claim 13 wherein the dew point in step i) is 15° C.

15. The process according to claim 13 wherein the coated potassium chloride granules of step iii) are essentially free of surfactants or processing aids and agents.

16. The process according to claim 13 wherein the alcohol is methyl alcohol.

17. The process according to claim 16 wherein the mixture of step ii) is about 10.3% ethylcellulose, 2.1% water and 87.6% methyl alcohol, by weight.

18. A method of manufacturing ethylcellulose-coated potassium chloride granules comprising the steps of:
i) forming a fluidized bed of potassium chloride crystals,
ii) spraying the fluidized crystals with a mixture consisting of ethylcellulose, alcohol, and sufficient water to control the buildup of static charge so as to enable substantially complete coating of the crystals, and
iii) drying the coated crystals to remove the water and alcohol to provide coated potassium chloride granules.

19. The method of claim 18 wherein the coated potassium chloride granules of step iii) are essentially free of surfactants or processing aids and agents.

20. The method of claim 18 wherein the mixture of step ii) comprises 0.5-2% water, by weight.

21. The method of claim 18 wherein the alcohol is methyl alcohol.

22. The method of claim 21 wherein the mixture of step ii) is about 10.3% ethylcellulose, 2.1% water and 87.6% methyl alcohol, by weight.
